(19) United States
(12) Patent Application Publication
(10) Pub. No.: US 2013/0098367 A1
(43) Pub. Date: Apr. 25, 2013

(54) Laryngeal Mask

(71) Applicant: Tien-Sheng Chen, Taipei City (TW)

(72) Inventor: Tien-Sheng Chen, Taipei City (TW)

(21) Appl. No.: 13/366,440

(22) Filed: Feb. 6, 2012

(30) Foreign Application Priority Data
Oct. 21, 2011 (TW) 100138333

(51) Int. Cl. A61M 16/04

(52) U.S. Cl. 128/207.15

(57) ABSTRACT

A laryngeal mask for covering an opening of the larynx and touching an opening of the esophagus of a patient is disclosed. The laryngeal mask has a tube and a mask portion, wherein the tube has a first end. The mask portion has an upper surface, a front end, and an esophageal blocking portion, wherein the upper surface connects to the first end; the esophageal blocking portion is situated at the front end. When the laryngeal mask is being placed into the patient's pharynx, the esophageal blocking portion moves along bilateral sides of the esophagus and blocks the opening of the esophagus such that the opening of the larynx is properly covered by the laryngeal mask.

### BACKGROUND OF THE INVENTION

1. Field of the Invention

The present invention relates to a medical device; more specifically, the present invention relates to a laryngeal mask.

2. Description of the Related Art

For apnea patients, the top priority for emergency medical personnel is to sustain the pulmonary gas supply. Among the various respiration sustaining instruments, the laryngeal mask airway (LMA) is a common alternative to endotracheal intubation due to its ease of operability. The laryngeal mask disclosed in U.S. Pat. No. 4,509,514 in 1985 is the original design of the most widely used laryngeal mask. However, this type of laryngeal mask is soft and is difficult to place into the throat of a patient due to its rounded front end. Therefore, laryngeal masks disclosed in U.S. Pat. No. 5,983,897 and U.S. Pat. No. 7,156,100 are modified versions of the laryngeal mask disclosed in 1985. Although the drawback of the U.S. Pat. No. 4,509,514 has been improved by modifying the rounded front ends to the pointed front end in U.S. Pat. No. 5,983,897 and U.S. Pat. No. 7,156,100, those two modified versions still have some inconvenience during operation; please refer to FIG. 1 and FIG. 1a for clear illustrations. The laryngeal mask disclosed in U.S. Pat. No. 5,983,897 and U.S. Pat. No. 7,156,100 are shown in FIG. 1. The widely-used laryngeal mask of the prior art laryngeal mask 100 comprises a laryngeal mask front end 110. When the prior art laryngeal mask 100 is being placed into the pharynx of a patient 80, the laryngeal mask front end 110 is curved and upward-wrapping by the extrusion between the laryngeal mask front end 110 and the laryngeal rear wall 81 (as shown in FIG. 1a). As a result, the prior art laryngeal mask 100 cannot be placed into the throat of the patient 80 properly because of the extrusion, which causes inconvenience to medical personnel. Furthermore, the pointed front end of the laryngeal mask front end 110 is difficult to turn within a human cavity and may damage the tissue of the upper respiratory tract of the patient.

Therefore, there is a need to provide a laryngeal mask to obviate the problem of the prior art.

### SUMMARY OF THE INVENTION

It is an object of the present invention to provide a laryngeal mask that can be placed into a patient's pharynx easily.

To achieve the abovementioned object, the laryngeal mask of the present invention is disclosed; the laryngeal mask covers the opening of the larynx of a patient and blocks the esophageal inlet of the patient. The laryngeal mask comprises a tube and a mask portion, wherein the tube comprises a first end. The mask portion comprises an upper surface, a front end, and an esophageal blocking portion, wherein the upper surface combines with the first end of the tube and the esophageal blocking portion is situated at the front end. The esophageal blocking portion further comprises two blocking portions and an indentation, wherein the indentation is situated between the two blocking portions, whereby when the laryngeal mask of the present invention enters the pharynx of the patient, the two blocking portions slide along the bilateral sides of the opening of the larynx and then block the esophageal inlet of the patient.

The laryngeal mask of the present invention employs the two protrusions situated on the bilateral sides of the esophageal blocking portion and an indentation situated between the two protrusions for smoothly entering the pharynx of the patient and for allowing the laryngeal mask to slide into the esophagus along the bilateral sides of the larynx and to block the esophageal inlet without damaging the trachea of the patient.

Because the structure of the present invention is novel and useful in application due to its improvement, the present invention is entitled to apply for a utility patent.

### BRIEF DESCRIPTION OF THE DRAWINGS

FIG. 1 is a schematic drawing illustrating the laryngeal mask of the prior art.

FIG. 1a is a schematic drawing illustrating the common error of the laryngeal mask of the prior art.

FIG. 2 is a schematic drawing illustrating one embodiment of the laryngeal mask of the present invention.

FIG. 3 is a bottom view schematic drawing illustrating one embodiment of the laryngeal mask of the present invention.

FIG. 4 is a top and partial sectional view illustrating one embodiment of the laryngeal mask of the present invention.

FIG. 5 is a top and partial sectional view illustrating another embodiment of the laryngeal mask of the present invention.

FIG. 5a is a cross-sectional view illustrated from the direction AA′ in FIG. 5.

FIG. 5b is a cross-sectional view illustrated form the direction BB′ in FIG. 5.

FIG. 6 is a schematic drawing illustrating one embodiment of the laryngeal mask of the present invention combined with a patient.

FIG. 6a is another schematic drawing illustrating one embodiment of the laryngeal mask of the present invention combined with a patient.

FIG. 6b is a further schematic drawing illustrating another embodiment of the laryngeal mask of the present invention combined with a patient.

### DETAILED DESCRIPTION OF THE PREFERRED EMBODIMENT

The advantages and innovative features of the invention will become more apparent from the following detailed descriptions when taken together with the accompanying drawings.

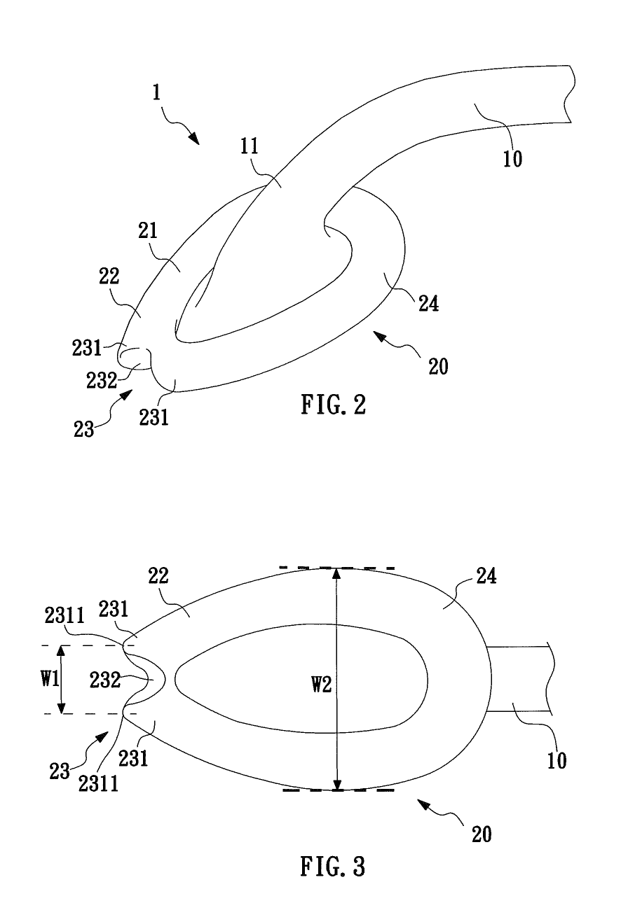

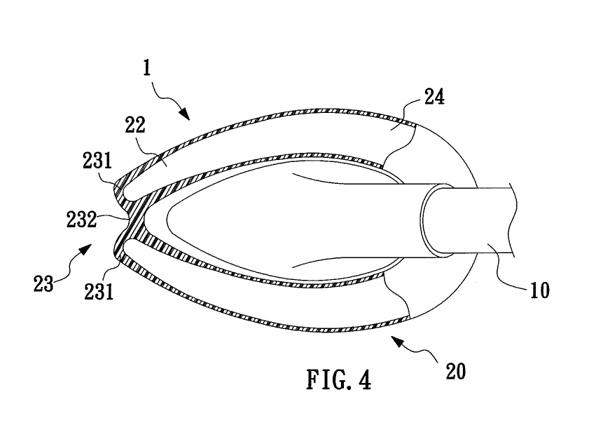

Please refer to FIG. 2 to FIG. 4, which are related to one embodiment of the laryngeal mask of the present invention, wherein FIG. 2 is a schematic drawing illustrating one embodiment of the laryngeal mask of the present invention; FIG. 3 is a bottom view schematic drawing illustrating one embodiment of the laryngeal mask of the present invention; FIG. 4 is a top and partial sectional view illustrating one embodiment of the laryngeal mask of the present invention.

As shown in FIG. 2, the laryngeal mask 1 of the present invention comprises a tube 10 and a mask portion 20, wherein the tube 10 comprises a first end 11, and the mask portion 20 comprises a top surface 21, a front end 22, an esophageal blocking portion 23, and a rear end 24. The upper surface 21 combines with the first end 11 of the tube 10. The esophageal blocking portion 23 is situated at the front end 22. As shown in FIG. 2 and FIG. 3, the esophageal blocking portion 23 further comprises two blocking portions 231 and an indentation 232; the indentation 232 is situated between the two blocking portions 231. As shown in FIG. 3, each of the two blocking portions 231 comprises a blocking portion front end 2311. It is noted that the size of the laryngeal mask 1 of the present invention may vary depending on the ages and the weights of different patients. While the laryngeal mask 1 of the present invention is entering a human's pharynx, in order to allow the two blocking portion front ends 2311 to slide along the bilateral sides of the larynx, as shown in FIG. 3, the front end width W1 between the two blocking portion front ends 2311 is substantially larger than the width of a human's pharynx, and the front end width W1 is the 20% to 50% of the largest width W2 of the mask portion 20. It is noted that, the ratio are not limited to the abovementioned range; i.e. the front end width W1 can be the 25% to 50% of the largest width W2 of the mask portion 20; or the front end width W1 can be the or 20% to 38% of the largest width W2 of the mask portion 20.

According to one embodiment of the present invention, as shown in FIG. 4, the mask portion 20 is a hollow ring structure for storing gases within. When a certain amount of gas is filled into the mask portion 20, a soft surface is formed on the outer side of the mask portion 20. It is noted that in order to maintain the shape of the two blocking portions 231 and the indentation 232, the width of the inner wall of the esophageal blocking portion 23 in this embodiment is greater than the width of the inner wall of the rear end 24 of the mask portion 20. Therefore, the specific shape of the esophageal blocking portion 23 can be maintained even before gas is filled, i.e., two protrusions at the bilateral sides and an indentation in the middle. Furthermore, the laryngeal mask 1 of this embodiment is made of plastic (PVC). However, the present invention is not limited to this; the laryngeal mask 1 can also made of silicon or rubber, and the structure of the mask portion 20 is not limited to a hollow mask. The mask portion 20 can be solid as well to increase the adaptability of the present invention.

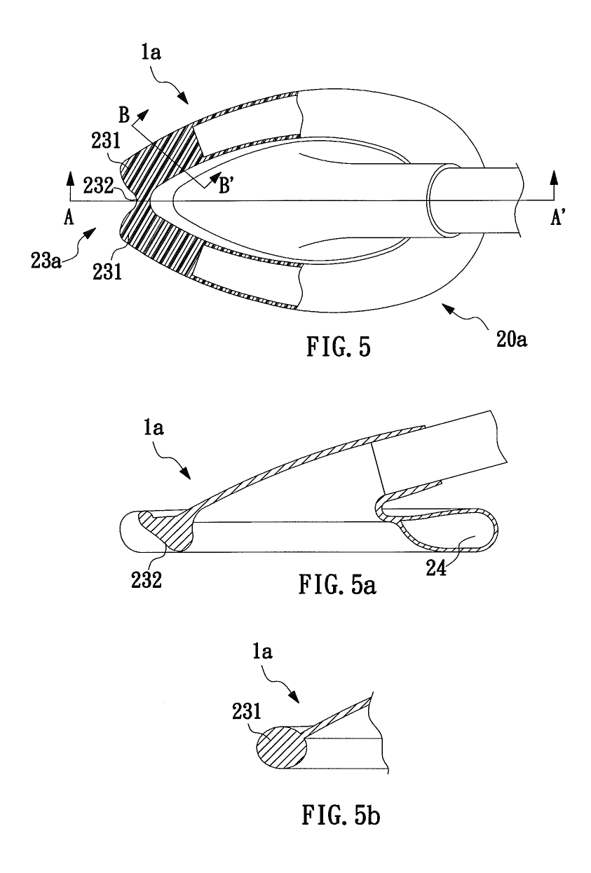

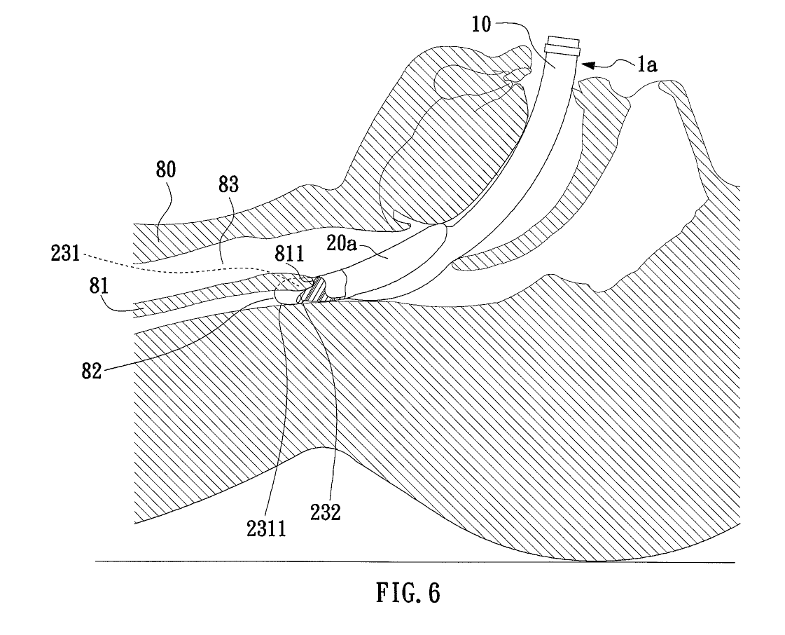

Please refer to FIG. 5, FIG. 5a, and FIG. 5b, wherein FIG. 5 is a top and partial sectional view illustrating another embodiment of the laryngeal mask of the present invention; FIG. 5a is a cross-sectional view illustrated from the direction AA′ in FIG. 5; FIG. 5b is a cross-sectional view illustrated from the direction BB′ in FIG. 5.

As shown in FIG. 5 and FIG. 5b, the most distinguishing difference between the laryngeal mask 1 of the present invention in this embodiment and the laryngeal mask 1a of the present invention is that the esophageal blocking portion 231 of the laryngeal mask 1a is solid. As illustrated in a cross-sectional view from the direction AA′ in FIG. 5a, the indentation 232 is an inclined surface. It is noted that according to one embodiment of the present invention, the rear end 24 of the mask portion 20 is a hollow mask portion. However, the present invention is not limited to this embodiment; the mask portion 20 can also be solid.

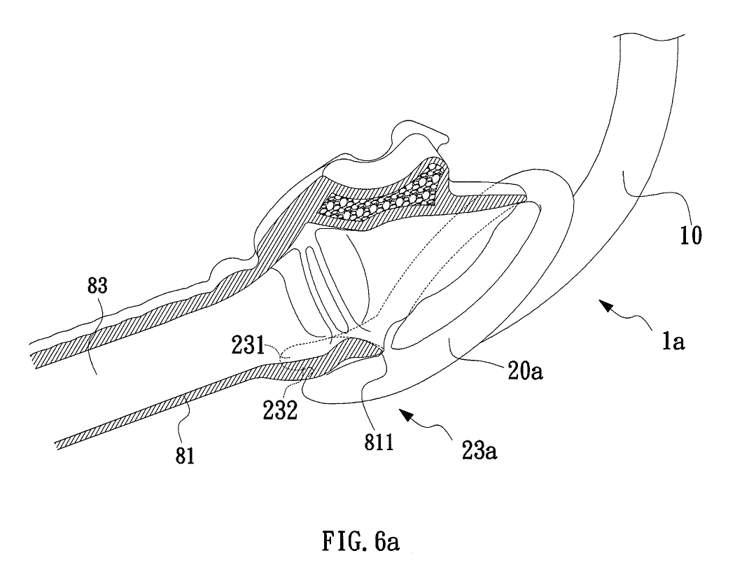

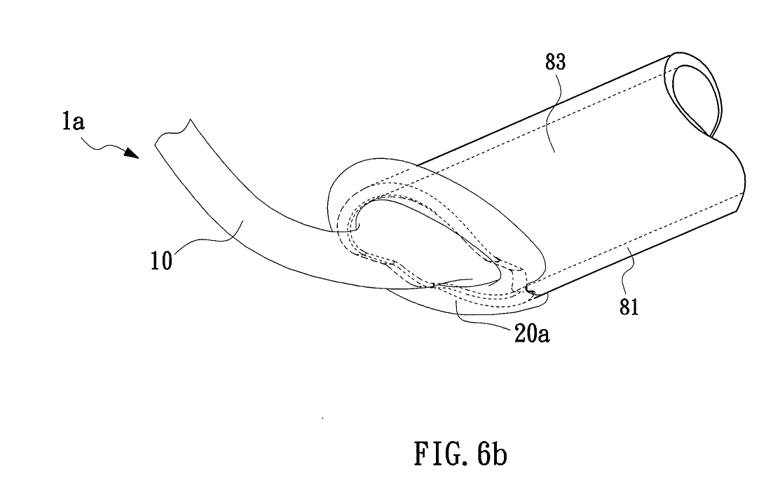

Please refer to FIG. 6, FIG. 6a, and FIG. 6b, wherein FIG. 6 is a schematic drawing illustrating one embodiment of the laryngeal mask of the present invention combined with a patient; FIG. 6a is another schematic drawing illustrating one embodiment of the laryngeal mask of the present invention combined with a patient; FIG. 6b is a further schematic drawing illustrating another embodiment of the laryngeal mask of the present invention combined with a patient.

As shown in FIG. 6, while the laryngeal mask 1 of the present invention is being used, the end of the laryngeal mask 1 with the mask portion 20 is placed into the pharynx of the patient 80 first by medical personnel. When the laryngeal mask 1 enters the pharynx of the patient 80, the two blocking portion front ends 2311 slide into the esophageal inlet 82 along the bilateral sides of the larynx 83 of the patient 80. Meanwhile, the esophageal blocking portion 23a touches the esophageal inlet 82 of the patient 80 and blocks the esophageal inlet 82, such that the two blocking portions 231 of the esophageal blocking portion 23a block the esophageal inlet 82 located at the bilateral sides of the larynx. When the laryngeal mask 1 enters the pharynx of the patient 80, as shown in FIG. 6 and FIG. 6a, the laryngeal rear wall 81 of the patient 80 touches the indentation 232. The indentation 232 in this embodiment is an inclined surface. After the two blocking portion front ends 2311 enter the esophageal inlet 82, the laryngeal rear wall 81 of the patient 80 is situated on the inclined surface and the indentation 232 touches the arytenoid 811 of the patient 80 at the same time. Meanwhile, the laryngeal mask 1 of the present invention forms an air-tight enclosure around the throat of the patient 80 for sheathing the opening of the larynx to form an airway (as shown in FIG. 6a and FIG. 6b).

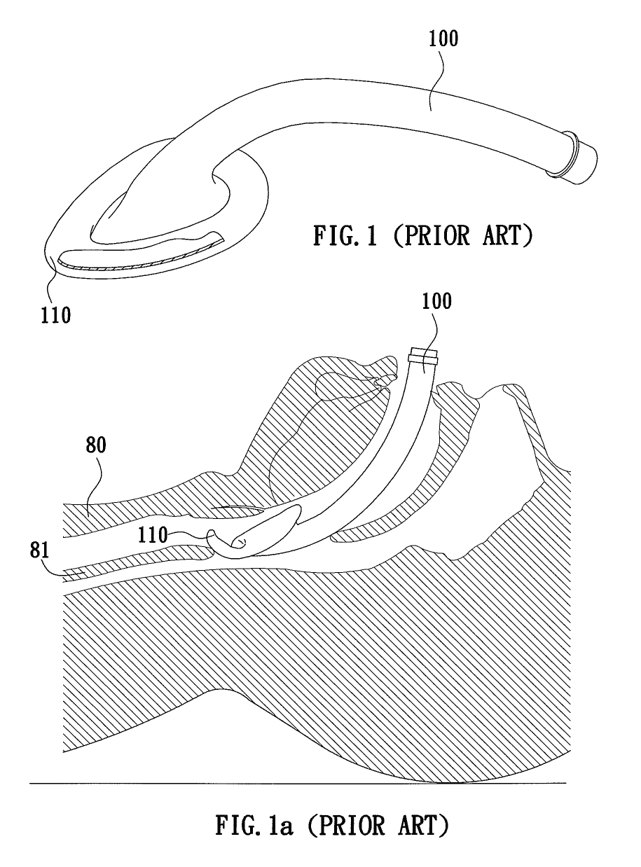

It is noted that the structure of the two blocking portions 231 and the indentation 232 of the esophageal blocking portions 23, 23a of the present invention matches the physical structure of the human larynx and the esophageal inlet such that the esophageal blocking portion 23 can slide into the esophageal inlet 82 along the bilateral sides of the larynx 83 of the patient 80. This kind of design can eliminate the drawback of the prior art laryngeal mask 100, i.e. preventing the laryngeal mask front end 110 from being curved and upward-wrapping by the extrusion between the laryngeal mask front end 110 and the laryngeal rear wall 81, which can cause failure in placement (as shown in FIG. 1a). In addition, because the esophageal blocking portion 23 has no sharp edges, damage to the tissue of the upper respiratory tract of patients during the placing the laryngeal mask 1 of the present invention can be reduced. Furthermore, after the laryngeal mask 1 of the present invention is placed, an external air vent can be connected to the tube 10 to allow oxygen to pass through the tube 10 and then to enter the trachea 83 to provide an oxygen supply for the patient 80. Because the esophageal inlet 82 of the patient 80 has been covered by the esophageal blocking portion 23, no air is allowed to enter the esophagus. Meanwhile, food inside the patient 80's stomach cannot backflow from the esophagus to the trachea thus cause no choking.

It must be noted that the above-mentioned embodiments are only for illustration. It is intended that the present invention cover modifications and variations of this invention provided that they fall within the scope of the following claims and their equivalents. Therefore, it will be apparent to those skilled in the art that various modifications can be made to the structure of the present invention without departing from the scope or spirit of the invention.

What is claimed is:

1. A laryngeal mask for covering an opening of the larynx of a patient and blocking an esophageal inlet of the patient, the laryngeal mask comprising:
a tube comprising a first end; and
a mask portion comprising:
an upper surface combined with the first end of the tube;
a front end; and
an esophageal blocking portion situated at the front end;
whereby, when the laryngeal mask enters into the pharynx of the patient, the esophageal blocking portion slides into the esophageal inlet of the patient along the bilateral sides of the opening of the larynx and blocks the esophageal inlet.

2. The laryngeal mask as claimed in claim 1, wherein the esophageal blocking portion further comprises two blocking portions and an indentation, wherein the indentation is situated between the two blocking portions, whereby when the laryngeal mask enters the pharynx of the patient, the two blocking portions slide along the bilateral sides of the opening of the larynx and cover the esophageal inlet.

3. The laryngeal mask as claimed in claim 2, wherein each of the two blocking portions comprises a blocking portion front end, and a width between the two blocking portion front end is 20% to 50% of the largest width of the mask portion.

4. The laryngeal mask as claimed in claim 3, wherein when the laryngeal mask enters the pharynx of the patient, the two blocking portion front ends slide along the bilateral sides of an opening of the larynx of the patient and enter the esophageal inlet.

5. The laryngeal mask as claimed in claim 4; wherein when the laryngeal mask enters the pharynx of the patient, the laryngeal rear wall of the patient touches the indentation.

6. The laryngeal mask as claimed in claim 5, wherein the indentation is an inclined surface; when the laryngeal mask enters the pharynx of the patient, the laryngeal rear wall of the patient is situated on the inclined surface.

7. The laryngeal mask as claimed in claim 6, wherein when the laryngeal mask enters the pharynx of the patient, the arytenoid of the laryngeal rear wall of the patient touches the indentation.

8. The laryngeal mask as claimed in claim 7, wherein the esophageal blocking portion is solid.

9. The laryngeal mask as claimed in claim 7, wherein the esophageal blocking portion is hollow.

10. The laryngeal mask as claimed in claim 9, wherein the mask portion further comprises a rear end, wherein the width of the inner wall of the esophageal blocking portion is thicker than the width of the inner wall of the rear end of the mask portion to maintain the shape of the mask portion even before the mask portion has been inflated.
